(19) United States
(12) Patent Application Publication
(10) Pub. No.: US 2015/0065806 A1
(43) Pub. Date: Mar. 5, 2015

(54) DOUBLE LUMEN ARTHROSCOPY PORT

(71) Applicant: Andrew Cooper, Salt Lake City, UT (US)

(72) Inventor: Andrew Cooper, Salt Lake City, UT (US)

(21) Appl. No.: 14/472,107

(22) Filed: Aug. 28, 2014

Related U.S. Application Data
(60) Provisional application No. 61/872,667, filed on Aug. 31, 2013.

(51) Int. Cl. A61B 17/34; A61B 17/32

(52) U.S. Cl. CPC A61B 17/3423; A61B 17/320016; A61B 2017/3445
USPC 600/204

(57) ABSTRACT

A device is provided to serve as a port for arthroscopic procedures. The device may include a cannula defining a lumen, with a partition extending the length of the cannula and dividing the lumen into two compartments. The surgeon may use one compartment for instrumentation such as an arthroscope or other surgical instruments, and the second compartment for suture and related suture instruments. This may enable the surgeon to keep untied sutures and the like separate from other surgical instruments and prevent tangling, etc.

### BACKGROUND OF THE INVENTION

1. The Field of the Invention

The present invention relates generally to instruments used for surgery. More specifically, the present invention relates arthroscopic, endoscopic, or other surgical port instruments.

2. State of the Art

Arthroscopic surgery is generally well known in the art, and is regularly employed by orthopedic surgeons to treat a wide variety of conditions on human joints. Arthroscopy is performed using a type of endoscope called an arthroscope, which is inserted into the joint through a small incision. The arthroscopic procedure may be performed to treat torn cartilage or ligaments, trim damaged cartilage, perform ACL reconstruction, etc. One advantage of arthroscopy that the joint does not have to be fully opened, but rather is accessed through small incisions, reducing recovery time and trauma to the connective tissue. However, the surgery is technically challenging because the surgeon must do all the work through the small incisions.

For example, shoulder arthroscopy typically requires placement of suture anchors and suture material within the shoulder joint. Typically, three 10-millimeter arthroscopy ports are inserted into incisions cut in the shoulder to provide the access necessary for the surgery. After the ports are placed in the shoulder joint, a high flow saline solution is then used to inflate the joint, washout any blood, and provide visibility for the surgeon. As sutures are placed, they are often left untied until all sutures are placed as desired. The untied suture is usually brought out through one of the ports to hold it in place while the next suture is placed.

Because the lumen of the port is also being used for instrumentation and cameras, the suture can become tangled, twisted, or damaged if it is brought out through the lumen of the port. Another option for the surgeon is to remove the port and bring the suture out through the access incision, and then reinsert the port. However, swelling and high flow saline can make this option messy and difficult. Thus, there is a need for a device to simplify the placement and management of suture material during arthroscopy and related surgeries.

### SUMMARY OF THE INVENTION

According to one aspect of the present disclosure, a surgical tool and method for use in arthroscopy is provided. The tool may comprise a port with at least one partition dividing the port into at least two compartments.

According to one configuration, the port may include a proximal end, through which instruments, suture, and the like are inserted. The port may also include a distal end comprising a hollow tubular member (or cannula) which is inserted into a patient, and the hollow tubular member may define a lumen.

According to another aspect of the present disclosure, the lumen may be divided into two or more compartments by a partition. The partition may be movable, allowing the surgeon to change or adjust the relative area of the compartments depending on the surgeon's needs. The surgeon may thus “select” between working in two different compartments. The partition may be formed of a material such that it is rigid, partly flexible, or fully flexible.

According to one configuration, the movable partition has a width slightly greater than the diameter of the cannula, such that the partition is naturally biased to bow towards one side of the cannula.

According to another aspect of the present disclosure, the partition may include a switch to assist the surgeon in switching the relative area of the compartments during surgery.

According to another aspect of the present disclosure, the proximal end of the device may be provided with a dam to prevent fluid outflow from the proximal end of the device. The dam may include an opening, such as a slit, to allow instrumentation, etc, to be inserted through the proximal end while minimizing fluid outflow.

According to another aspect of the present disclosure, the compartments of the port may be in fluid connection. According to one configuration, there may be an opening in the one or more partitions to allow the space on either side of the partition to be in fluid connection.

According to another aspect of the present disclosure, the port may include a connector for connection to a saline line for fluid.

According to another aspect of the present disclosure, a method is provided wherein a surgeon may insert a port as described herein, and use the two or more compartments of the port to manage instrumentation and sutures during surgery.

These and other aspects of the present invention are realized in an arthroscopy port and method of use as shown and described in the following figures and related description. It will be appreciated that various embodiments of the invention may not include each aspect set forth above and aspects discussed above shall not be read into the claims unless specifically described therein.

### BRIEF DESCRIPTION OF THE DRAWINGS

Various embodiments of the present disclosure are shown and described in reference to the numbered drawings wherein:

FIG. 1 shows a perspective view of an arthroscope port according to the prior art;

FIG. 2 shows a perspective view of an arthroscope port as provided in the current disclosure;

FIG. 3 shows a cut-away, partially cross-sectional view of the arthroscope port of FIG. 2 taken along line III of FIG. 2;

FIG. 4 shows a perspective view of another possible configuration of an arthroscope port as described herein;

FIG. 5A shows a distal end-view of a hollow tubular member of an arthroscope as described herein, with the movable partition in a center position;

FIG. 5B shows a distal end-view of the hollow tubular member of the arthroscope of FIG. 5A, with the movable partition in an off-center position;

FIG. 6A shows a distal end-view of a configuration of a hollow tubular member of an arthroscope as described herein, with the movable partition in a center position;

FIG. 6B shows a distal end view of the hollow tubular member of the arthroscope of FIG. 6A, with the movable partition in an off-center position;

FIG. 7A shows a distal end-view of a configuration of a hollow tubular member of an arthroscope as described herein, with the movable partition in a center position;

FIG. 7B shows a distal end-view of the hollow tubular member of the arthroscope of FIG. 7A, with the movable partition in an off-center position;

FIG. 8A shows a distal end-view of a configuration of a hollow tubular member of an arthroscope as described herein, with the movable partition in a center position;

FIG. 8B shows a distal end-view of the hollow tubular member of the arthroscope of FIG. 8A, with the movable partition in an off-center position;

FIG. 9A shows a distal end-view of a configuration of a hollow tubular member of an arthroscope as described herein, with the movable partition in an off-center position; and

FIG. 9B shows a distal end-view of the hollow tubular member of the arthroscope of FIG. 9A, with the movable partition in another off-center position.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity. Similarly, not every embodiment need accomplish all advantages of the present invention.

### DETAILED DESCRIPTION

The invention and accompanying drawings will now be discussed in reference to the numerals provided therein so as to enable one skilled in the art to practice the present invention. The skilled artisan will understand, however, that the methods described below can be practiced without employing these specific details, or that they can be used for purposes other than those described herein. Indeed, they can be modified and can be used in conjunction with products and techniques known to those of skill in the art in light of the present disclosure. For example, while the description often discusses applications for arthroscopic surgery, the technique is not limited to that field and may apply to other types of surgery as well. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

Reference in the specification to “one embodiment,” “one configuration,” “an embodiment,” or “a configuration” means that a particular feature, structure, or characteristic described in connection with the embodiment may be included in at least one embodiment, etc. The appearances of the phrase “in one embodiment” in various places may not necessarily limit the inclusion of a particular element of the invention to a single embodiment, rather the element may be included in other or all embodiments discussed herein.

Furthermore, the described features, structures, or characteristics of embodiments of the present disclosure may be combined in any suitable manner in one or more embodiments. In the following description, numerous specific details are provided, such as examples of products or manufacturing techniques that may be used, to provide a thorough understanding of embodiments of the invention. One skilled in the relevant art will recognize, however, that embodiments discussed in the disclosure may be practiced without one or more of the specific details, or with other methods, components, materials, and so forth. In other instances, well-known structures, materials, or operations may not be shown or described in detail to avoid obscuring aspects of the invention.

Before the present invention is disclosed and described in detail, it should be understood that the present invention is not limited to any particular structures, process steps, or materials discussed or disclosed herein, but is extended to include equivalents thereof as would be recognized by those of ordinarily skill in the relevant art. More specifically, the invention is defined by the terms set forth in the claims. It should also be understood that terminology contained herein is used for the purpose of describing particular aspects of the invention only and is not intended to limit the invention to the aspects or embodiments shown unless expressly indicated as such. Likewise, the discussion of any particular aspect of the invention is not to be understood as a requirement that such aspect is required to be present apart from an express inclusion of the aspect in the claims.

It should also be noted that, as used in this specification and the appended claims, singular forms such as “a,” “an,” and “the” may include the plural unless the context clearly dictates otherwise. Thus, for example, reference to “a spring” may include an embodiment having one or more of such springs, and reference to “the layer” may include reference to one or more of such layers.

As used herein, the term “substantially” refers to the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing the nearly all of the length of a lumen would be substantially enclosed, even if the distal end of the structure enclosing the lumen had a slit or channel formed along a portion thereof. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Distal and proximal, as used herein, are from the perspective of the person using the arthroscopy port. Thus, proximal means nearer to the user and distal means farther from the person using the port. The port described herein thus has a distal end which is inserted into a patient and a proximal end that remains outside the patient, as described below.

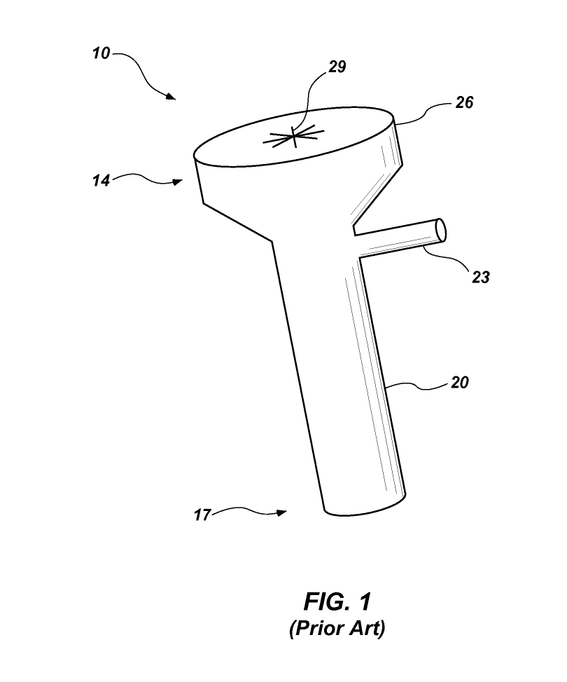

Turning now to FIG. 1, there is shown a prior art port for use in arthroscopy, generally indicated at 10. The port has a proximal end 14, which remains outside the body, and a distal end 17, which may be inserted into a joint space through an incision. The port 10 includes a hollow tubular member 20 (i.e., a cannula). The hollow tubular member 20 or cannula defines a lumen through which the surgeon inserts instrumentation, etc. The proximal end 14 may also include a connector 23 to provide connection for saline tubing, as saline is often used in arthroscopy to inflate the joint. The proximal end 14 may also include a dam 26. The dam 26 is integral to the proximal end 14 and used to prevent or minimize fluid outflow from the proximal end 14 of the port. The dam 26 is often formed of silicone rubber and includes a slit-like opening 29 at the top in to allow instrumentation, etc. to be inserted through the proximal end 14 of the port 10 while minimizing fluid outflow. The port 10 also generally includes a trocar (not shown in FIG. 1) with a tip that is placed in the lumen during port placement to penetrate soft tissues around the joint. The trocar may be of any type suitable, and may have a tip that is rigid, semi-rigid, rounded, sharp, etc. Once the port 10 is in place, the trocar may be removed.

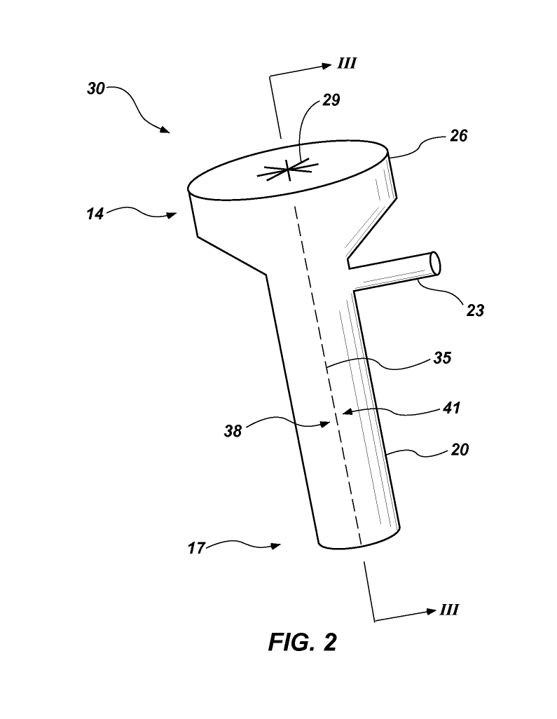

Turning now to FIG. 2, there is shown a configuration of an arthroscopy port according to the current disclosure, generally indicated at 30. This port 30 may include a hollow tubular member 20 that defines a lumen 40. The port may also include one or more internal movable partitions 35, as indicated by the dashed line in FIG. 2, within the hollow tubular member 20. The movable partition 35 may divide the lumen into separate compartments. According to the exemplary configuration shown in FIG. 2, one movable partition 35 is provided and the lumen 40 is divided into a first compartment 38 and a second compartment 41 on opposing sides of the partition 35. The first compartment 38, by way of example, may be used for access for surgical instruments and the like, while the second compartment 41 may be used for access to sutures, etc.

The partition 35 may be movable such that the relative area of the compartments may be adjusted during surgery depending on the surgeons needs. Or, in other words, the surgeon may select between the compartments during surgery. For example, the surgeon may desire to use instrumentation and thus move the partition 35 such that the first compartment 38 has a large relative area and the second compartment 41 has a small relative area. Then, a surgeon may desire to place a suture and move the partition 35 such that the first compartment 38 has a small relative area and the second compartment 41 has a large relative area. The surgeon may thus move the partition 35 back and forth as desired during surgery to facilitate management of suture and instrumentation (discussed in more detail below).

The partition 35 may be formed of rigid, semi-rigid, or flexible material. The material used to construct the movable partition 35 may be any type of suitable material known in the art, and may be a material that is strong enough so as to not be damaged by sharp instruments passing through the port. The material may be thin and flexible, while still being resistant to tears and punctures. For example, the partition 35 may be formed of a rigid or semi-rigid plastic suspended on an elastomer membrane, such as silicone. Another example may be a partition formed of a flexible sheet that does not stretch, such as teflon, poly-propylene, Kevlar, nylon, polyethylene, etc. Still another example may be a partition formed of the desired material and then coated with a puncture/cut-resistant material.

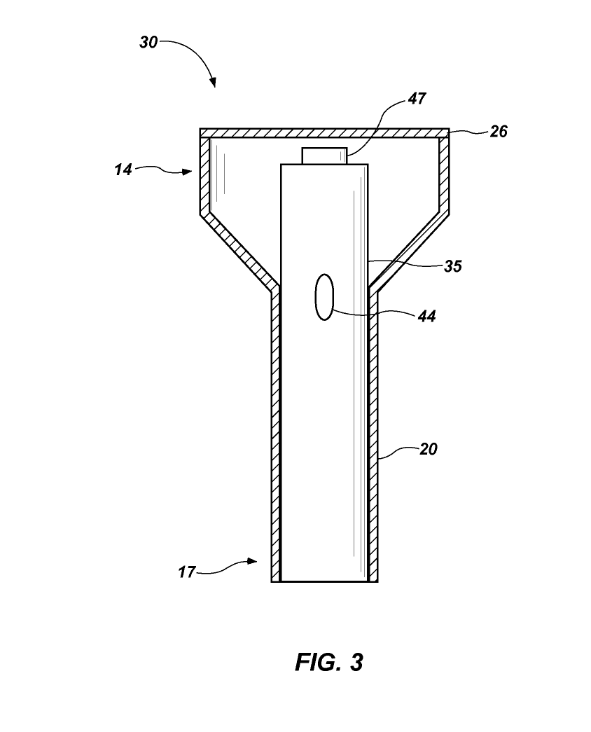

Turning now to FIG. 3, there is shown a front, cross-sectional view (taken along line III of FIG. 2) of the arthroscopy port shown in FIG. 2. The movable partition 35 may be provided with an opening 44 such that the first compartment 38 and the second compartment 41 are in fluid connection. This may prevent water pressure from blocking the movement of the movable partition 35. One having skill in the art will appreciate there are numerous ways to allow for the first compartment 38 and the second compartment 41 to be in fluid connection; for example, by way of a smaller opening, a larger opening, multiple openings, etc. Any of these configurations could be used and would be within the scope of the current disclosure.

The movable partition 35 may be connected to the sidewalls of the hollow tubular member 20 for the length of the hollow tubular member 20. The movable partition 35 may extend the length of the hollow tubular member 20 and then terminate, or it may be configured to extend further proximally, as shown in FIG. 3. According to the configuration shown in FIG. 3, the movable partition 35 extends for substantially the entire length of the hollow tubular member 20. In other configurations, the movable partition 35 may be configured such that it extends for only part of the length of the hollow tubular member 20.

The movable partition 35 may also be provided with a projection 47 near the proximal end 14 of the port 30. This projection 47 could serve as a manual switch to assist the surgeon in moving the movable partition 35 during surgery. This projection 47 as shown in FIG. 3 extends vertically from the movable partition 35 and may be accessed through the opening 29 in the dam 26 at the proximal end 14 of the port 30. The projection 47 may also be configured to extend horizontally from the movable partition 35 and may be accessed through a separate opening provided on the side of the port 30 rather than through the top of the dam 26. The surgeon may also move the movable partition 35 during surgery without the use of a manual switch, for example, by putting horizontal pressure on movable partition 35.

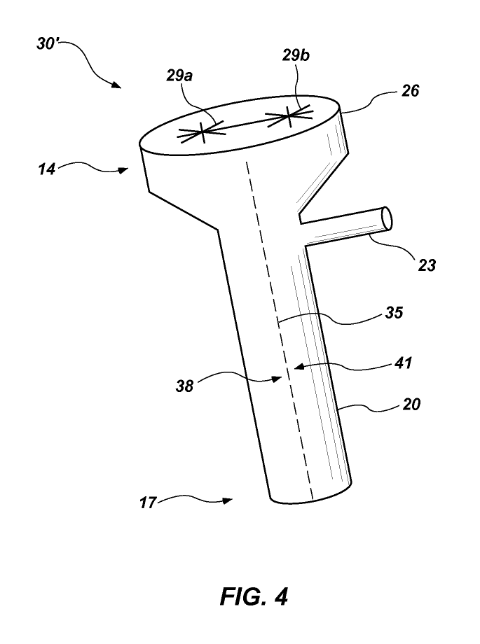

Turning now to FIG. 4, there is shown a perspective view of another possible configuration of an arthroscopy port, generally indicated at 30′. According to this configuration, two slit-like openings 29a, 29b may be provided in the dam 26. Thus, if a surgeon desired to access the first compartment 38, instrumentation and the like may be inserted through the opening 29a in the dam 26. If the surgeon desired to access the second compartment 41, instrumentation and the like may be inserted through the opening 29b in the dam 26 (or, by way of example, sutures taken up through the second compartment 41 may be pulled through the opening 29b). One having skill in the art will appreciate that the dam 26 could have other configurations for the opening 29, such as a single slit, one or more X-shaped slits, or various other shapes. Additionally, if multiple openings 29a,b are provided, the openings may be connected/joined, as shown in FIG. 4, or may be spatially separate.

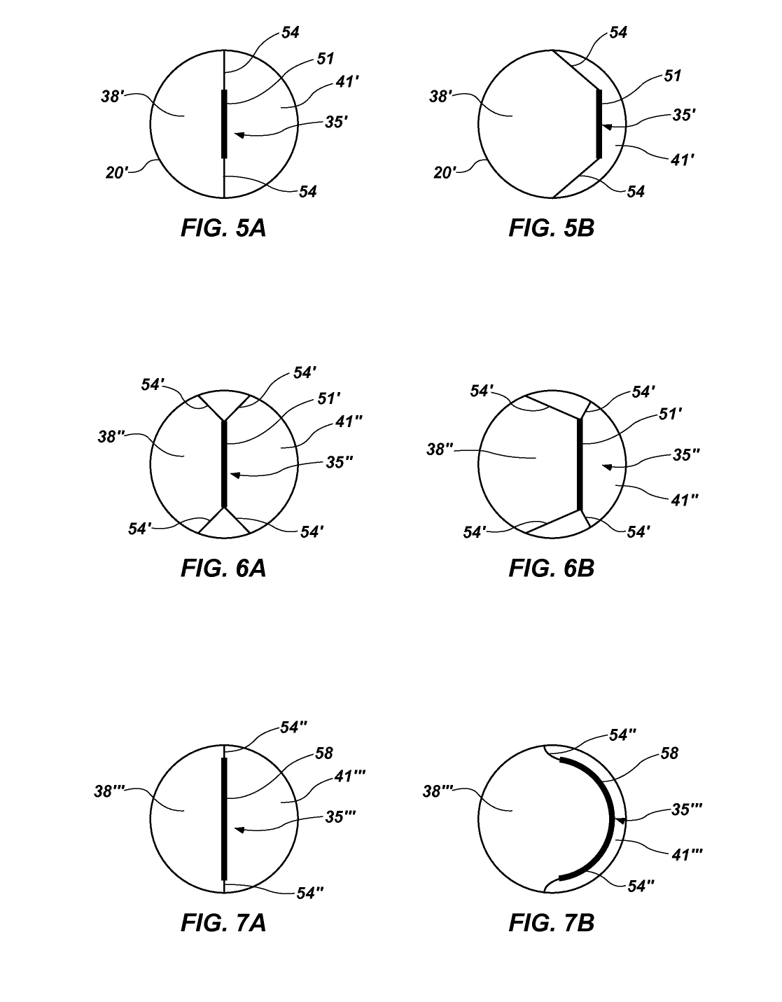

Various configurations are possible for the movable partition 35. For example, FIGS. 5A,B through 9A,B show distal end views of the hollow tubular member 20 of arthroscopy ports as disclosed herein with various configurations of the movable partitions. These are configurations that are shown by way of example, and one with skill in the art will appreciate that numerous other configurations are possible and are within the scope of the present disclosure.

FIG. 5A shows a port having an exterior wall forming a hollow tubular member 20′, and a movable partition 35′ which divides the hollow tubular member, or cannula, 20′ into a first compartment 38′ and a second compartment 41′. The movable partition 35′ may be constructed of a rigid material 51 (as indicated by the thicker solid line) suspended on an elastic membrane 54 (as indicated by the thinner solid line). (It will be appreciated that the varying thickness of the lines in FIGS. 5A-7B is to demonstrate the different materials, and is not necessarily indicative of the thicknesses of the actual materials).

As seen in FIG. 5B, the partition 35′ may be moved from its center position by the flexibility of the elastic membrane 54. This movement of the partition 35′ increases the relative area of the first compartment 38′, while decreasing the area of the second compartment 41′. As described in detail below, this may be the position of the partition that the surgeon may prefer while primarily using the arthroscope/endoscope and other instrumentation during surgery. In this position, the surgeon is not working with sutures, and the sutures need very little space to pass through the port. When the surgeon desires to work with the sutures, such as when the surgeon may place a suture, the partition 35′ may be moved towards the other side of the hollow tubular member, or cannula, 20′. This movement of the partition 35′ decreases the relative area of the first compartment 38′, while increasing the area of the second compartment 41′. In this manner, the surgeon may select between working with instruments and working with sutures, while keeping the sutures and instrumentation from becoming entangled.

According to FIGS. 6A and 6B, there is shown another possible configuration for the movable partition 35″ of the port. This partition 35″ is also formed of rigid material 51′ (as indicated by the thicker solid line) mounted on an elastic membrane 54′ (as indicated by the thinner solid line) as in FIGS. 5A and 5B, but may now be provided with two elastic membrane connections to the hollow tubular member on both ends of the rigid material. FIG. 6A shows this configuration with the movable partition 35″ in the center position, while FIG. 6B shows this configuration with the movable partition 35″ moved to the right side, increasing the relative area of the first compartment 38″, while decreasing the area of the second compartment 41″. It will be appreciated that the movable partition 35″ could also be moved to the left of center to decrease the relative area of the first compartment 38″ while increasing the area of the second compartment 41″. A surgeon may thus move the partition 35″ to each side and thereby select which compartment to work with at a given time.

FIGS. 7A and 7B show another distal-end view of a possible configuration for the movable partition 35 of the port. According to this configuration, the movable partition 35′″ may be formed of a semi-flexible material 58 (as indicated by the thicker solid line) suspended on an elastic membrane 54″ (as indicated by the thinner solid line). FIG. 7A shows the movable partition 35″′ in a center position, and FIG. 7B shows the movable partition 35″′ moved to the right side, with the semi-flexible material 58 in a flexed position.

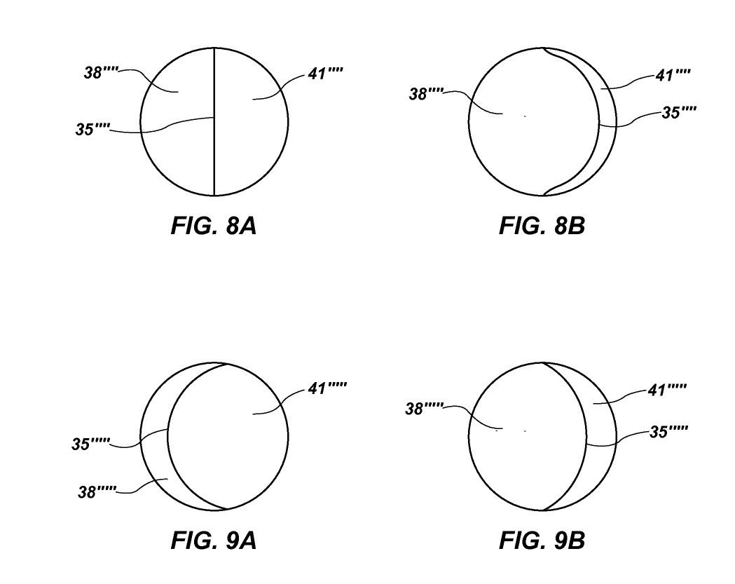

FIGS. 8A and 8B show yet another distal-end view of a possible configuration for the movable partition 35″″ in a port 30. According to this configuration, the movable partition 35″″ may be fully formed from a flexible material. FIG. 8A shows the movable partition in a center position, and FIG. 8B shows the movable partition moved to the right side.

FIGS. 9A and 9B show distal-end views of yet another possible configuration for the movable partition 35′″″. This view shows the width of the movable partition, and in this particular exemplary configuration, the width of the movable partition is slightly greater than the diameter of the cannula. The movable partition may also be formed of a material that is at least partially flexible. Thus, the partition having a slightly greater width than the diameter of the cannula may cause the partition to bow to one side naturally. By pushing the partition, it can be made to bow to the other side. FIG. 9A shows the partition 35′″″ bowed to the left side (the surgeon is thus selecting use of the right compartment 41′″″), and FIG. 9B shows the partition 35″″′ bowed to the right side (with the surgeon now selecting use of the left compartment 38′″″).

For example, the partition 35 could have a width slightly greater than the diameter of the cannula 20 in an ambient state. The partition 35 could be made of a partially flexible or fully flexible material (such as a plastic) which would cause the partition to bow to either side, and the surgeon could push on the outerwall of the port to make the outerwall temporarily distend to allow the partition to be pushed past the center line, causing the partition to bow in the other direction. The surgeon could also push the actual partition (either directly, or with an instrument) to cause it to bow in the other direction.

In use, the patient may be prepared and draped in the usual way. The surgeon may establish the port in the joint using a trocar, and then remove the trocar and connect saline tubing to the connector 23. The surgeon may then insert instrumentation, such as an arthroscope or endoscope, into the first compartment 38 of the port 30. This step may include, for example, inserting the instrumentation through the proper slit above the first compartment of the port in the dam at the top of the port, or by switching the projection on the movable partition to increase the relative area of the first compartment of the port, or by pushing on the movable partition with the instrumentation to increase the relative area of the first compartment of the port, etc.

A typical arthroscope may measure about 5 millimeters to 15 millimeters in diameter, and may be up to 25 millimeters in diameter for laparoscopic procedures. The lumen of the hollow tubular member may be about 10 millimeters in diameter, by way of example. One having skill in the art would appreciate that the port as described herein may be made with a lumen of a larger diameter or a smaller diameter. Ports with lumens of different diameters may be manufactured such that a surgeon may choose a port with the appropriate-sized lumen based on the patient's and surgeon's needs.

Any additional smaller instrumentation that may be needed by the surgeon may also be placed through the first compartment 38 of the port 30. As the surgeon places suture during the procedure, the surgeon may bring out the ends of the untied suture through the second compartment 41 of the port to hold them in place while the next suture is placed. These untied ends may all be brought out through the second compartment of the port until all sutures are placed as desired.

The arthroscope/endoscope and any other additional instrumentation may remain in the first compartment, separated from the suture of the second compartment by the movable partition. This may keep the suture from being entangled in or damaged by the instrumentation moving in and out of the port as well as eliminating the need for removal of the port. Additionally, the small size of the suture may allow for nearly full access to the port through the first compartment 38 while the suture is sequestered in its own compartment 41. When the surgeon is ready to tie all sutures, instrumentation in the first compartment 38 may be removed and the partition 35 may be moved towards the first compartment to increase the relative area of the second compartment 41 (thereby decreasing the relative area of the first compartment). The surgeon us thus provided with nearly full access to the port through the second compartment 41 for additional space for tying of sutures. Depending on the surgery performed and the needs of the surgeon, the partition may be easily moved several times during surgery. This may allow the surgeon the maximum space to perform the necessary work, while also keeping instrumentation and sutures organized.

A port for use with an endoscope surgical instrument is described herein and the port may include: a hollow tubular member defining a lumen having an open distal end and a proximal end, and at least one partition dividing the lumen into a first compartment and a second compartment. The first compartment and the second compartment may be in fluid connection, and the partition may be movable.

The partition may be formed from a piece of plastic material having a length greater than an internal diameter of the hollow tubular member, the plastic material having at least one flexible portion so that the plastic material is biased to extend toward one side, and the plastic material being sufficiently flexible that it can be pushed toward an opposite side of the hollow tubular member and remain biased toward the opposite side.

The partition may include a projection configured to assist in moving the partition. The partition may be at least partly flexible. The partition may be fully flexible. The port may further include a dam covering the proximal end. The dam may include at least one opening. The dam may include two openings. The port may include a connector configured to receive saline tubing. The hollow tubular member may have an inside and an outside, the outside of the hollow tubular member being threaded.

A port for use in arthroscopy may include: a hollow tube adapted to receive an endoscope, the tube having an open distal end sized for insertion into a patient, a proximal end covered by a dam, and a movable partition configured to divide the hollow tube into a first compartment and a second compartment, the first compartment and second compartment being in fluid connection. The dam of the port may include at least one slit-like opening configured to receive surgical instruments while minimizing fluid outflow. The dam may include two slit-like openings configured to receive surgical instruments while minimizing fluid outflow. The movable partition may include at least one opening in the partition.

A method for performing arthroscopic surgery is described herein, and the method may include inserting a port into an incision, the port having a cannula with an open distal end and a proximal end having a dam with an openable portion, the cannula defining a lumen extending between the distal end and the proximal end, and at least one movable partition dividing the lumen into a first compartment and a second compartment, the first and second compartments being in fluid connection; inserting instrumentation through the at least one openable portion of the dam and into the first compartment of the lumen; placing sutures having untied ends; bringing the untied ends of the sutures through the open distal end of the port into the second compartment of the cannula.

According to the method for performing arthroscopic surgery, the dam may include a first slit-like opening generally above the first compartment and a second slit-like opening generally above the second compartment, and wherein the step of inserting instrumentation further includes inserting the instrumentation through the first slit-like opening.

The method may also include the step of bringing the untied ends of the sutures further includes bring the untied ends of the sutures through the second slit-like opening. The method above may also comprise the movable partition further including a projection configured to assist in moving the partition, and wherein the first compartment has a relative area and the second compartment has a relative area, and the method further includes the step of applying pressure to the projection such that the movable partition is moved to increase the relative area of the first compartment and decrease the relative area of the second compartment.

A method for conducting an arthroscopic procedure is described herein, and the method may include inserting a port into an incision, the port having an outer wall defining a lumen, an open distal end in communication with the lumen and a selectively openable dam adjacent a proximal end of the lumen, the port further having a movable partition dividing the lumen into a first compartment and a second compartment; advancing an instrument through the first compartment and performing a part of the procedure; and advancing an instrument through the second compartment and performing part of the procedure, wherein the movable partition is moved toward the first compartment to increase the size of the second compartment prior to or while inserting an instrument through the second compartment. The method may also include the step of positioning untied sutures in the first compartment prior to moving the partition.

There is thus disclosed an improved arthroscopy port and method of use. It will be appreciated that numerous changes may be made to the present invention without departing from the scope of the claims.

What is claimed is:

1. A port for use with an endoscope surgical instrument including:
a hollow tubular member defining a lumen having an open distal end and a proximal end, and at least one movable partition dividing the lumen into a first compartment and a second compartment.

2. The port of claim 1, wherein the first compartment and the second compartment are in fluid connection.

3. The port of claim 1, wherein the lumen has a diameter and the movable partition has a width, the width of the movable partition being greater than the diameter of the lumen, and wherein the movable partition is formed of a material that is sufficiently flexible that it can be pushed toward a side of the hollow tubular member and remain biased toward the side.

5. The port of claim 1, wherein the partition includes a projection configured to assist in moving the partition.

6. The port of claim 1, wherein the partition is at least partly flexible.

7. The port of claim 1, wherein the partition is fully flexible.

8. The port of claim 1, further including a dam covering the proximal end.

9. The port of claim 7, wherein the dam includes at least one opening.

10. The port of claim 7, wherein the dam includes two openings.

11. The port of claim 1, wherein the port further includes a connector configured to receive saline tubing.

12. The port of claim 1, wherein the hollow tubular member has an inside and an outside, the outside of the hollow tubular member being threaded.

13. A port for use in arthroscopy, including:
a hollow tube adapted to receive an endoscope, the tube having an open distal end sized for insertion into a patient, a proximal end covered by a dam, and a movable partition configured to divide the hollow tube into a first compartment and a second compartment, the first compartment and second compartment being in fluid connection.

14. The port of claim 13, wherein the dam includes at least one slit-like opening configured to receive surgical instruments while minimizing fluid outflow.

15. The port of claim 13, wherein the dam includes two slit-like openings configured to receive surgical instruments while minimizing fluid outflow.

16. The port of claim 13, wherein the movable partition includes at least one opening in the partition.

17. A method for performing arthroscopic surgery, the method including:
inserting a port into an incision, the port having a cannula with an open distal end and a proximal end having a dam with an openable portion, the cannula defining a lumen extending between the distal end and the proximal end, and at least one movable partition dividing the lumen into a first compartment and a second compartment, the first and second compartments being in fluid connection;
inserting instrumentation through the at least one openable portion of the dam and into the first compartment of the lumen;
placing sutures having untied ends;
bringing the untied ends of the sutures through the open distal end of the port into one of the first compartment and the second compartment of the cannula; and
passing an instrument through the other compartment of the first compartment and the second compartment not having the untied ends.

17. The method for performing arthroscopic surgery according to claim 16, wherein the dam includes a first slit-like opening generally above the first compartment and a second slit-like opening generally above the second compartment, and wherein the step of inserting instrumentation further includes inserting the instrumentation through the first slit-like opening.

18. The method according to claim 16, wherein the step of bringing the untied ends of the sutures further includes bring the untied ends of the sutures through the second slit-like opening.

19. The method according to claim 18, wherein the movable partition further includes a projection configured to assist in moving the partition, and wherein the first compartment has a relative area and the second compartment has a relative area, and the method further includes the step of applying pressure to the projection such that the movable partition is moved to increase the relative area of the first compartment and decrease the relative area of the second compartment.

20. A method for conducting an arthroscopic procedure, the method comprising:
inserting a port into an incision, the port having an outer wall defining a lumen, an open distal end in communication with the lumen and a selectively openable dam adjacent a proximal end of the lumen, the port further having a movable partition dividing the lumen into a first compartment and a second compartment;
advancing an instrument through the first compartment and performing a part of the procedure; and
advancing an instrument through the second compartment and performing part of the procedure, wherein the movable partition is moved toward the first compartment to increase the size of the second compartment prior to or while inserting an instrument through the second compartment.

21. The method of claim 20, wherein untied sutures are positioned in the first compartment prior to moving the partition.
